SEQUENCE LISTING (1 sequence)

SEQ ID NO: 1 - protein, 736 aa, Bacillus anthracis
MEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

(19) United States
(12) Patent Application Publication
(10) Pub. No.: US 2010/0183675 A1
(43) Pub. Date: Jul. 22, 2010

(54) Stable vaccine compositions and methods of use

(76) Inventors: Allan Watkinson, Guisborough (GB); Matthew Duchars, Annapolis, MD (US)

Correspondence Address: Alan J. Grant, Esq.;c/o Carella, Byrne, Bain, Gilfillan, Cecchi,, Stewart & Olstein, 5 Becker Farm Road, Roseland, NJ, 07068, US

(21) Appl. No.: 12/321,564

(22) Filed: Jan. 22, 2009

(51) Int. Cl. A61K 39/07

(52) U.S. Cl. 424/246.1

(57) ABSTRACT

A stable lyophilized protein formulation containing recombinant Protective Antigen (rPA) is described that can be reconstituted with a suitable diluent to generate a high potency protein concentration reconstituted formulation which is suitable for use as a vaccine against anthrax infection in mammals, especially human beings. These formulations were prepared by lyophilizing rPA in the presence of a reducing sugar, such as trehalose, and methods of such lyophilization are described. The resulting lyophilized mixture or composition is subsequently reconstituted to high immunogenicity without apparent loss of stability of the rPA protein. Methods of using said formulations in vaccination are also described.

### FIELD OF THE INVENTION

The present invention relates generally to the field of protein storage, especially following industrial scale preparation and where said proteins have form part of a vaccine composition intended for administration to mammals, especially human beings.

### BACKGROUND OF THE INVENTION

Vaccines are commonly formulated using an adjuvant. A common adjuvant-type is the aluminium based colloids, usually refered to as alum. More specifically these are usually aluminium hydroxide (aluminium oxyhydroxide) (also called alhydrogel) and aluminium phosphate (also called adju-phos). For example, vaccines useful against organisms such as anthrax (Bacillus anthracis) are commonly formulated with alhydrogel, which binds the anthrax antigen used in such vaccines (so called subunit vaccines).

In the recent past, advances in vaccine therapy have made it possible to produce a variety of proteins for use in vaccines, such as use of recombinant Protective Antigen (rPA) from anthrax, using recombinant DNA technology. Because of the relatively large size and complex nature of proteins generally, the formulation of such proteins in vaccines poses some unique problems. To remain antigenically active, a protein formulation must preserve the conformational integrity of the protein representing the basic antigenic structure in the vaccine. Degradation of proteins can involve chemical instability (for example, where bond formation or cleavage results in a new antigenic structure) or physical instability (for example, changes in the higher order structure of the protein). Physical instability can result from, for example, denaturation or aggregation of the protein(s) present in the vaccines while chemical instability can result from chemical reactions that either break the amino acid chain of the protein or serve to modify one or more amino acid side groups present on the protein (which groups may be essential for immunogenic activity).

Lyophilization (commonly termed “freeze-drying”) is a well known technique for preserving proteins and operates by removing water from the protein composition of interest. It is a process by which the material to be dried is first frozen and then the frozen solvent (ice in the case of water) is removed by sublimation under high vacuum. Additives may be included in pre-lyophilized formulations to enhance stability during the freeze-drying process and/or to improve stability of the lyophilized product upon storage. Unfortunately, such procedures have heretofore proved unreliable for the lyophilization and storage of certain antigenic proteins useful in vaccines, especially for anthrax vaccines, where stability of the lyophilized powder has resulted in significant loss of activity following reconstitution.

The present invention solves at least some of the problems heretofore encountered with lyophilization of protein formulations, especially of formulations containing recombinant protective antigen (rPA) of anthrax (Bacillus anthracis), by providing a formulation that retains antigenic activity and thus utility as a vaccine even with long storage in the lyophilized state, so that the reconstituted vaccine composition is still highly useful for protection of mammals, such as human being, against infection by anthrax bacilli.

### BRIEF SUMMARY OF THE INVENTION

In one aspect, the present invention relates to a stable reconstituted formulation or vaccine composition, which may be isotonic, comprising an antigen or immunogen for use in a vaccine, preferably an anthrax antigen for use in a vaccine against anthrax, more preferably anthrax Protective Antigen, and most preferably a recombinant Protective Antigen (rPA), in an amount of about 50 μg/mL to about 400 μg/mL, preferably about 200 μg/ml, and a pharmaceutically acceptable carrier, wherein this formulation has been reconstituted from a lyophilized mixture or composition of the anthrax, or other, antigen. In one embodiment, the reconstituted formulation may contain trehalose, such as trehalose in an amount that provided lyoprotection of the lyophilized mixture or composition.

In preferred embodiments of the reconstituted formulation, said formulation contains a salt, such as phosphate or NaCl or both, a detergent, such as Tween, and an adjuvant, such as alhydrogel (alum), chitosan, MPL or CpG. In preferred embodiments, the adjuvant is CpG1018 or alhydrogel or, especially preferred, both.

The vaccine according to the present invention may comprise adjuvants additional to alum. Examples of such adjuvants are well known in the art and include oligonucleotides, especially so-called “CpG” oligonucleotides, especially phosphorothioate oligonucleotides, most preferably oligonucleotides which target Toll Like Receptor 9 receptors. Other additional adjuvants that may be present include saponins, PCPP polymer, lipopolysaccharide derivatives, MPL [Monophosphoryl Lipid A], MDP muramyl di-peptide (MDP), t-MDP, Flagellin and flagellin-fusion proteins, IC31, OM-174 and Leishmania elongation factor, ISCOMS, chitosan, SB-AS2, AS02, SB-AS4, non-ionic block copolymers and SAF [Syntex Adjuvant Formulation].

When an additional adjuvant is employed, the additional adjuvant is preferably incorporated with the stabilised sub-unit vaccine although the additional adjuvant may alternatively be present in the separate adjuvant and/or diluent composition.

In certain embodiments, the stable sub-unit vaccine composition or formulation may also incorporate an agent to reduce or prevent agglomeration during preservation, for example a small amount of a non-ionic surfactant, especially an ethoxylated sorbitan ester, for example polyoxyethylenesorbilan monolaurate, typically comprising about 20 ethyleneoxy units.

In another aspect, the present invention relates to a method for preparing a stable lyophilized vaccine formulation, comprising the steps of:

(a) forming a composition of an anthrax antigen containing rPA and a lyoprotective amount of a reducing sugar; and

(b) freeze-drying said composition to form a stable lyophilized vaccine formulation.

In preferred embodiments, the mixture or composition to be lyophilized also contains a buffer, such as phosphate, a salt, such as NaCl, a detergent, such as Tween, and an adjuvant, such as alhydrogel (alum), chitosan, mpl or CpG, with CpG1018 and alhydrogel being especially preferred.

In one embodiment, the lyophilized composition also comprises a bulking agent added in an amount sufficient to provide mass to the lyophilized composition and contribute to the physical structure of the lyophilized bulk material.

In a further aspect, the present invention relates to a kit comprising a container that holds a lyophilized mixture or composition of an anthrax, or other, vaccine containing rPA, trehalose and a pharmaceutically acceptable carrier; and a set of instructions for reconstituting the lyophilized composition with a diluent to an rPA concentration of about 200 μg/mL. In one embodiment, a diluent is provided with the kit. Preferably, the diluent contains an adjuvant, such as alhydrogel.

The present invention also relates to a method of protecting against, or treating, a microbial, especially a bacterial, infection in a mammal, comprising administering to a mammal either at risk of developing or afflicted with such infection a therapeutically-effective amount of the vaccine composition of the invention. In further embodiments thereof, the bacterial infection is an anthrax (Bacillus anthracis) infection and/or the mammal is a human being.

### BRIEF DESCRIPTION OF THE DRAWINGS

FIG. 1 shows the amino acid sequence of a recombinant Protective Antigen (rPA) from Bacillus anthracis (anthrax).

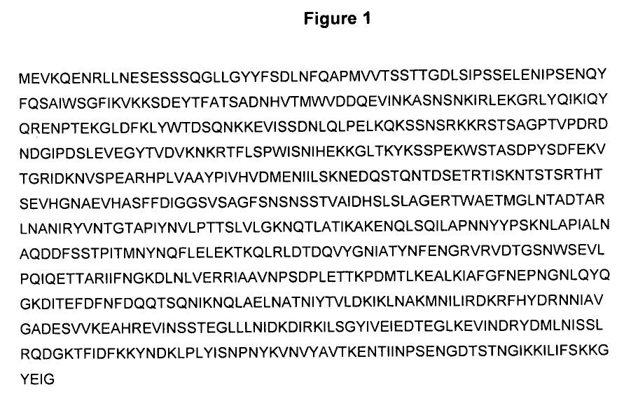

### DEFINITIONS

As used herein, a “stable” formulation is one wherein the antigenic protein, such as rPA, retains its physical and chemical stability and integrity upon storage, such as in the form of a lyophilized powder, solid or cake. Analytical techniques for measuring protein stability are known in the art and will not be described in detail herein. See, for example, Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). For example, a “stable” formulation may be one wherein less than about 10% and preferably less than about 5% of the protein is present as an aggregate in the formulation. In other embodiments, any increase in aggregate formation following lyophilization and storage of the lyophilized formulation can be determined. For example, a “stable” lyophilized formulation may be one wherein the increase in aggregate in the lyoplilized formulation is less than about 5% and preferably less than about 3%, when the lyophilized formulation is stored at 2-8° C. for at least 6 months, preferably 9 months, more preferably up to one year or longer, such as at least about 16 months.

As used herein, the term “about” means an amount that does not substantially reduce the effectiveness of the component whose range or amount is being recited by more than 10%. Wherever an amount or range is recited using the term “about” it is to be understood that the invention specifically contemplates the same range or amount as if the term “about” had not been used. Thus, “in about the range of” and “in the range of” are alternative embodiments of the methods and formulations of the invention.

As used herein, a “reconstituted” formulation is one that has been prepared by dissolving a lyophilized protein formulation in a diluent such that the protein is dispersed in the reconstituted formulation. The reconstituted formulation in suitable for administration to a patient.

As used herein “isotonic” refers to a formulation or composition of the invention having substantially the same osmotic pressure as human blood. Isotonic formulations will generally have an osmotic pressure from about 250 to 350 mOsm/Kg. Isotonicity can be measured using a vapor pressure or ice-freezing type osmometer, for example.

As used herein, the term “lyoprotectant” means a molecule that, when combined with an immunogenic protein, such as rPA, of the invention, substantially reduces, if not prevents, chemical and/or physical instability of said protein upon lyophilization and subsequent storage, sufficient to maintain the immunogenic properties of the protein and thereby insure continued utility as a vaccine. Examples of such lyoprotectants include non-reducing sugars, such as sucrose or trehalose, amino acids, such as monosodium glutamate or histidine, methylamines like betaine, lyotropic salts, like magnesium sulfate, polyols, like trihydric or higher sugar alcohols, including glycerin, erythritol, glycerol, arabitol, xylitol, sorbitol, and manmitol, glycols, like propylene glycol and polyethylene glycol, and combinations of any or all of these.

As used herein, the term “lyoprotecting amount” refers to the amount of a lyoprotectant, for example, trehalose, that when added to an immunogen, such as rPA, of the invention, substantially preserves the physical and chemical stability and integrity of the immunogen upon lyophilization and storage so that, once reconstituted, said immunogen retains its immunogenic properties sufficiently to permit its use in a vaccine.

As used herein, the term “diluent” means a carrier that is pharmaceutically acceptable (safe and non-toxic for administration to a human) and is useful for the preparation of a reconstituted formulation. Exemplary diluents include sterile water, bacteriostatic water for injection (BWFI), a pH buffered solution (e.g. phosphate-buffered saline), sterile saline solution, Ringer's solution or dextrose solution.

As used herein, the term “preservative” means a compound that can be added to the diluent to essentially reduce bacterial action in the reconstituted formulation, thus facilitating the production of a multi-use reconstituted formulation, for example. Examples include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, 2-phenoxyethanol, thimerosal, benzethonium chloride, formaldehyde, butyl and benzyl alcohol, allyl parabens such as methyl or propyl paraben, catechol, resorcinol, cyclohexanol, 3-pentanol, and m-cresol. The most preferred preservative herein is 2-phenoxyethanol.

As used herein, the term “bulking agent” refers to a compound that adds mass to the lyophilized mixture or composition and contributes to the physical structure of the lyophilized cake (e.g. facilitates the production of an essentially uniform lyophilized cake which maintains an open pore structure). Exemplary bulking agents include mannitol, glycine, dextran, polyethylene glycol and xorbitol. “Treatment” refers to both therapeutic treatment and prophylactic or preventative measures. Those in need of treatment include those already with the disorder as well as those in which the disorder is to be prevented.

As used herein the term “effective amount” or “therapeutically effective amount” means a dosage sufficient to treat, inhibit, or alleviate one or more symptoms of a bacterial infection, especially anthrax infection. The precise dosage may vary with such factors as the age, immune system status, general health, and environmental circumstances of the patient, the nature and extent of the infection (or anticipated infection) and the availability of subsequent treatment/vaccination.

### DETAILED DESCRIPTION OF THE INVENTION

The antigen to be formulated is generally prepared using techniques well known in the art, including isolation from the organism as well as synthesis by recombinant technology or direct chemical synthesis of the polypeptide used in the antigen. The Protective Antigen used in the present invention was prepared by recombinant technology and is referred to herein as recombinant Protective Antigen (rPA).

In accordance with the present invention, there is provided a stable reconstituted formulation, comprising a sub-unit vaccine antigen, for example, an anti-microbial antigen, such as an anthrax antigen containing recombinant Protective Antigen (rPA), and a pharmaceutically acceptable carrier, which reconstituted formulation has been prepared from a lyophilized composition of said anthrax antigen and a lyoprotective amount of trehalose. In separate and preferred embodiments of such formulation or composition, intended for use as a vaccine, rPA is present in the reconstituted formulation in the range of about 100 to 300 μg/ml, preferably about 150 to 250 μg/ml, most preferably about 200 μg/ml.

The reconstituted anthrax vaccine formulation of the invention may also contain trehalose. In one embodiment, the trehalose is present in the reconstituted formulation in the range of about 3% to 7%, preferably in the range of about 4% to 6%, more preferably in the range of about 4.5% to 5.5%, and most preferably at about 5% (w/v).

The stable reconstituted vaccine formulation of the invention is one reconstituted from a lyophilized composition that was maintained at a temperature of at least 5° C. for a period of at least 1 month prior to reconstitution, or at least 25° C. for a period of at least 1 month prior to reconstitution, or wherein said temperature is at least 30° C., 35° C., 40° C., 45° C., 50° C., 55° C., 60° C., 65° C., 70° C., or even higher, and for periods of at least 1 month, 2 months, 3 months, 4 months, 5 months, 6 months, 7, months, 8 months, 9 months, 10 months, or even longer, such as a year, or 16 months or at least 2 years.

The reconstituted anthrax vaccine formulation of the invention may also contain a buffer. In specific embodiments thereof, the buffer (or salt) is phosphate at a concentration of in the range of 3 mM to 7 mM, preferably in the range of 4 mM to 6 mM, more preferably in the range of 4.5 mM to 5.5 mM, with concentrations of about 4 mM and about 5.25 mM being especially preferred. In other preferred embodiments, the reconstituted formulation comprises NaCl, for example, at about 0.39%. The reconstituted formulation of the invention may also contain a detergent, such as Tween 20. In one specific but non-limiting example thereof Tween 20 is present at about 0.02%.

The reconstituted anthrax vaccine formulation of the invention may also contain an adjuvant, especially one selected from the group alhydrogel (alum), chitosan, mpl and CpG, with CpG and anhydrogel preferred. In one embodiment, the adjuvant is CpG1018, for example, present at about 2 mg/ml. In another embodiment, the adjuvant is alhydrogel, for example, present at about 0.26% (w/v).

A complete but non-limiting example of such a formulation is one that contains 200 μg/ml rPA, 2 mg/ml CpG1018, 5% trehalose, 5.25 mM phosphate, 0.39% NaCl, 0.02% Tween 20, 0.26% w/v alhydrogel (see Example 1). In one embodiment of a vaccine formulation of the invention, the reconstituted formulation is isotonic.

The present invention also provides a method for preparing a stable reconstituted formulation, comprising the steps of:

(a) lyophilizing a mixture or composition of a sub-unit vaccine antigen, for example, an anti-microbial antigen, such as an anthrax antigen containing rPA, and a lyoprotective amount of trehalose; and

(b) reconstituting the lyophilized mixture or composition of step (a) in a diluent such that the reconstituted formulation is stable and has an rPA concentration of between about 0.1 and 10 times the rPA concentration in the mixture or composition before lyophilization.

In other examples of the methods of the invention, the diluent used in step (b) for reconstituting the mixture of step (a) comprises an adjuvant, preferably selected from alhydrogel (alum), chitosan, MPL or CpG, most preferably anhydrogel. In non-limiting examples, anhydrogel is present in the range of about 0.2% to about 0.3%, or is present in the range of about 0.25% to about 0.3%, or preferably is present at about 0.26%.

In other such examples, the diluent comprises a salt, such as phosphate and/or NaCl. In selected examples, the phosphate is present in the range of about 3 to 7 mM, or is present in the range of about 4 to 6 mM, or is present in the range of about 4.5 to 5.5 mM, or the phosphate is preferably present at about 5 mM. In other such examples, the NaCl is present in the range of about 0.3% to about 0.5%, or is present in the range of about 0.35% to about 0.45%, or is preferably present at about 0.39%. In a preferred embodiment, the diluent has a pH about 7.4.

In a preferred embodiment, the diluent contains 0.26% alhydrogel in 5 mM phosphate and 0.39% NaCl, pH 7.4.

Sub-unit vaccines containing anti-microbial antigens and which can be employed are usually recombinant protein-based antigens. Examples of such anti-microbial antigens include Hepatitis B protective antigens, Herpes Simplex Virus antigens, Influenza antigens, Congenital cytomegalovirus (CMV) antigens, Tuberculosis antigens, HIV antigens, Diphtheria antigens, Tetanus antigens, Pertussis antigens and Yersinia pestis protective antigens, such as antigens comprising one, two or more antigenic proteins, for example those disclosed in patent application WO96/28551, incorporated herein by reference. Most preferably, the antigen is an anthrax protective antigen, such as recombinant protective antigen (rPA), especially that having the amino acid sequence of SEQ ID NO: 1.

The present invention further provides a method for preparing a stable lyophilized vaccine formulation, comprising the steps of:

(a) forming a mixture or composition of a sub-unit vaccine antigen, for example, an anti-microbial antigen, such as anthrax antigen containing rPA, and a lyoprotective amount of a reducing sugar; and

(b) freeze-drying said mixture or composition to form a stable lyophilized vaccine formulation.

In specific examples of any of the methods of the invention, the composition of step (a) contains rPA in the range of about 200 to 600 μg/ml, or about 300 to 500 μg/ml, or about 350 to 450 μg/ml, or preferably at about 400 μg/ml. In other examples of the methods of the invention, the composition of step (a) contains trehalose is present in the range of about 2 to 20%, or in the range of about 4 to 18%, or about 6 to 16%, or about 8 to 14%, or about 8 to 12%, or about 9 to 12%, or preferably at about 10% (w/v).

In other embodiments of the method, the composition or mixture of step (a) contains a salt, preferably phosphate and/or NaCl (or saline). In examples thereof, the phosphate is present in the range of about 0.3 to 0.7 mM, or in the range of about 0.35 to 0.65 mM, or about 0.4 to 0.6 mM, or about 0.45 to 0.55 mM, or preferably is present at about 0.5 mM. In additional such examples, the NaCl is present in the range of about 7 to 13 mM, or about 8 to 12 mM, or about 9 to 11 mM, or about 9.5 to 10.5 mM, or preferably is present at about 10 mM.

In other examples of the methods of the invention, the mixture or composition of step (a) contains an adjuvant, preferably wherein the adjuvant is selected from the group consisting of alhydrogel (alum), chitosan, mpl or CpG. In preferred embodiments thereof, the adjuvant is CpG, such as CpG1018. In examples thereof, CpG1018 is present in the range of about 2 mg/ml to about 6 mg/ml, or in the range of about 3 mg/ml to about 5 mg/ml, or in the range of about 3.5 mg/ml to about 4.5 mg/ml, or CpG1018 is preferably present at about 4 mg/ml.

Examples of suitable oligonucleotide compounds which may be employed as additional adjuvants include those described in U.S. Pat. No. 6,207,648, U.S. Pat. No. 6,589,940, U.S. Pat. No. 7,255,868 and U.S. Pat. No. 7,276,489 (the disclosures of which are incorporated herein by reference).

In certain embodiments, the stabilized vaccine composition may also incorporate an agent to reduce or prevent agglomeration during preservation, for example a small amount of a non-ionic surfactant, especially an ethoxylated sorbitan ester, for example polyoxyethylenesorbilan monolaurate, typically comprising about 20 ethyleneoxy units.

In additional examples of the method, the mixture or composition of step (a) further comprises a detergent, for example, Tween 20. In non-limiting examples, Tween 20 is present in the range of about 0.3% to about 0.5%, or is present in the range of about 0.35% to about 0.45%, or preferably is present at about 0.04%.

In an especially preferred embodiment of the methods of the invention, the mixture or composition in step (a) comprises 400 μg/ml rPA, 4 mg/ml CpG1018, 10% trehalose, 0.5 mM phosphate, 10 mM NaCl, and 0.04% Tween 20.

The present invention, also provides for a composition suitable for lyophilization, comprising an anthrax antigen containing recombinant Protective Antigen (rPA) and a lyoprotective amount of trehalose. In non-limiting embodiments of this composition, rPA is present in the range of about 100 μg/mL to about 700 μg/mL, or is present in the range of about 200 to 600 μg/ml, or is present in the range of about 250 to 550 μg/ml, or is present in the range of about 300 to 500 μg/ml, or is present in the range of about 350 to 450 μg/ml, is preferably present at about 400 μg/ml. In other examples of the methods of the invention, the mixture or composition of step (a) contains trehalose is present in the range of about 2 to 20%, or in the range of about 4 to 18%, or about 6 to 16%, or about 8 to 14%, or about 9 to 12%, or preferably at about 10% (w/v).

In keeping with the invention, the lyoprotectant is added to the pre-lyophilized formulation or composition to be lyophilized. In preferred embodiments, this additive is a non-reducing sugar, for example, sucrose or trehalose, the latter being especially preferred. In one embodiment, the amount of lyoprotectant added is such that, upon reconstitution, the resulting formulation is isotonic. In addition, the amount of sugar added must be sufficient to prevent the undesirable degradation or aggregation, for example, of the antigenic protein, such as rPA, often occurring on lyophilization of such proteins.

In preferred embodiments of the invention, it may be desirable to add a surfactant, detergent, to the pre-lyophilized composition and/or the reconstituted formulation. Examples of useful surfactants include nonionic surfactants such as polysorbates (e.g. polysorbates 20 (or Tween 20) or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palnidopropyl-, or isostearamidopropyl-betaine (e.g lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethyl glycol, polypropyl glycol, and copolymers of ethylene and propylene glycol (e.g. Pluronics, PF68 and the like). Tween 20 is a preferred surfactant or detergent. The amount added is sufficient to reduce aggregation of the reconstituted protein and minimizes the formation of particulates after reconstitution. For example, the surfactant or detergent may be present in the pre-lyophilized composition in an amount from about 0.001-0.5%, and preferably from about 0.005-0.05%.

In specific embodiments of the invention, the lyoprotectant (such as trehalose) may be used in combination with a bulking agent (e.g. mannitol or glycine) to prepare the pre-lyophilization formulation. Use of such bulking agents may facilitate production of a more uniform lyophilized cake (i.e., without excessive pockets and the like).

In other embodiments, the composition contains a salt, preferably phosphate and/or NaCl (or saline). In examples thereof, the phosphate is present in the range of about 0.3 to 0.7 mM, or in the range of about 0.35 to 0.65 mM, or about 0.4 to 0.6 mM, or about 0.45 to 0.55 mM, or preferably is present at about 0.5 mM. In additional such examples, the NaCl is present in the range of about 7 to 13 mM, or about 8 to 12 mM, or about 9 to 11 mM, or about 9.5 to 10.5 mM, or preferably is present at about 10 mM.

In other examples, the composition contains an adjuvant, preferably wherein the adjuvant is selected from the group consisting of alhydrogel (alum), chitosan, mpl or CpG. In preferred embodiments thereof, the adjuvant is CpG, such as CpG1018. In examples thereof, CpG1018 is present in the range of about 2 mg/ml to about 6 mg/ml, or in the range of about 3 mg/ml to about 5 mg/ml, or in the range of about 3.5 mg/ml to about 4.5 mg/ml, or CpG1018 is preferably present at about 4 mg/ml.

In additional examples, the composition of further comprises a detergent, for example, Tween 20. In non-limiting examples, Tween 20 is present in the range of about 0.3% to about 0.5%, or is present in the range of about 0.35% to about 0.45%, or preferably is present at about 0.04%.

In an especially preferred embodiment, the composition suitable for lyophilization comprises 400 μg/ml rPA, 4 mg/ml CpG1018, 10% trehalose, 0.5 mM phosphate, 10 mM NaCl, and 0.04% Tween 20.

The present invention also provides a method for preparing such a composition, said method comprising essentially the procedure of step (a) recited elsewhere herein for methods of the invention.

The phosphate present in said composition to be lyophilized can serve the purposes of buffering the composition as well as the vaccine formulation produced after reconstitution. However, phosphate is not intended as a limiting buffer and other buffers may be used either in place or phosphate or in combination with it. Examples of such buffers include histidine, phosphate, Tris, citrate, succinate and other organic acids. The buffer concentration can be from about 1 mM to about 20 mM, or from about 3 mM to about 15 mM, depending, for example, on the buffer and the desired isotonicity of the vaccine formulation (e.g. of the reconstituted formulation). Such buffers may also provide a lyoprotective effect in addition to that of trehalose.

The present invention also provides a kit comprising:

(a) a container that holds a lyophilized composition of an anthrax vaccine containing rPA, trehalose and a pharmaceutically acceptable carrier; and

(b) instructions for reconstituting the lyophilized composition with a diluent to an rPA concentration of about 200 μg/mL.

Such a kit may further comprise a container of a diluent suitable for reconstituting the lyophilized composition. Specific embodiments of such diluent are as described elsewhere herein for the diluent used in the methods of the invention.

The kits of the invention commonly contain the lyophilized composition in a vial or other suitable container, including, for example, bottles, vials (e.g. dual chamber vials), syringes (such as dual chamber syringes) and test tubes. The container may be formed from a variety of materials such as glass or plastic. The container holds the lyophilized formulation and the label on, or associated with, the container may indicate directions for reconstitution and/or use. For example, the label may indicate that the lyophilized formulation is reconstituted to protein concentrations as described above. The label may further indicate that the formulation is useful or intended for subcutaneous administration. The container holding the formulation may be a multi-use vial, which allows for repeat administrations of the reconstituted formulation. The kit, which is an article of manufacture, may further comprise a second container comprising a suitable diluent of a type as disclosed herein. Upon mixing of the diluent and the lyophilized formulation, the final rPA concentration in the reconstituted formulation will generally be in a range as disclosed herein, but generally at about 200 μg/ml. The kit may further include other materials desirable from a commercial and user standpoint, including other buffers, diluents, filters, needles, syringes, and package inserts with instructions for use.

Generally, vaccines are prepared as injectables, in the form of aqueous solutions or suspensions. Vaccines in an oil base are also well known such as for inhaling. Solid forms which are dissolved or suspended prior to use may also be formulated. Pharmaceutically acceptable carriers, diluents and excipients are generally added that are compatible with the active ingredients and acceptable for pharmaceutical use.

The pharmaceutical compositions and/or formulations useful herein contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids such as water, saline, glycerol and ethanol, and the like, including carriers useful in forming sprays for nasal and other respiratory tract delivery or for delivery to the ophthalmic system. A thorough discussion of pharmaceutically acceptable carriers, diluents, and other excipients is presented in REMINGTON'S PHARMACEUTICAL SCIENCES (Mack Pub. Co., N.J. current edition). Those described elsewhere herein are especially preferred for use in the formulations, compositions, methods and kits of the invention.

Vaccine compositions may further incorporate additional substances to stabilize pH, or to function as adjuvants, wetting agents, or emulsifying agents, which can serve to improve the effectiveness of the vaccine.

Vaccines are generally formulated for parenteral administration and are injected either subcutaneously or intramuscularly. Such vaccines can also be formulated as suppositories or for oral administration, using methods known in the art, or for administration through nasal or respiratory routes.

The amount of vaccine sufficient to confer immunity to pathogenic bacteria, viruses, or other microbes is determined by methods well known to those skilled in the art in view of the guidance provided herein. This quantity will be determined based upon the characteristics of the vaccine recipient and the level of immunity required (as already noted above). Where vaccines are administered by subcutaneous or intramuscular injection, a range of 0.5 to 500 μg purified protein may be given. As useful in the present invention, such dosages are commonly sufficient to provide about 1 μg, possibly 10 μg, even 50 μg, and as much as 100 μg, up to 500 μg of immunogenic protein, or immunogenic polypeptide, or immunogenically active fragments thereof. In addition, more than one such active material may be present in the vaccine. Thus, more than one antigenic structure may be used in formulating the vaccine, or vaccine composition to use in the methods disclosed herein. This may include two or more individually immunogenic proteins or polypeptides, proteins or polypeptides showing immunogenic activity only when in combination, either quantitatively equal in their respective concentrations or formulated to be present in some ratio, either definite or indefinite.

A vaccine composition for use in the processes disclosed herein may include one or more immunogenic proteins, one or more immunogenic polypeptides, and/or one or more immunogenically active immunogens comprising antigenic fragments of said immunogenic proteins and polypeptides, the latter fragments being present in any proportions selected by the use of the present invention. The exact components, and their respective quantities, making up the vaccines, and vaccine compositions, useful in the methods of the present invention are determined, inter alia, by the nature of the disease to be treated or prevented, the severity of such condition where it already exists, the age, sex, and general health of the recipient, as well the personal and professional experience and inclinations of the researcher and/or clinician utilizing these methods.

For a vaccine of the present invention, such as a sub-unit vaccine for use against anthrax, specific non-limiting examples of this vaccine composition are those wherein said rPA is present at about 10 to 300 μg/ml, preferably 50 to 300 μg/ml, or wherein said rPA is present at about 100 to 300 μg/ml, more preferably about 150 to 250 μg/ml, and most preferably wherein said rPA is present at about 200 μg/ml.

In other examples, the dose of antigen, preferably rPA, to be administered is at least about 5 μg, or at least about 10 μg, or at least about 25 μg, or at least about 50 μg, or at least about 75 μg, or at least about 100 μg, or at least about 150 μg, or at least about 200 μg, with a preferred dose of about 100 μg. A preferred dose volume is about 0.5 ml.

The present invention further relates to a vaccine comprising a purified antigen, preferably an anthrax antigen, more preferably anthrax protective antigen (PA) and most preferably a recombinant anthrax protective antigen (rPA), such as that described in WO 2007/122373, pub. 1 Nov. 2007, the disclosure of which is hereby incorporated by reference in its entirety) bound to an adjuvant, preferably an alum-based adjuvant. In a preferred embodiment, the present invention relates to a sub-unit vaccine comprising alum-bound rPA, at least about 2 mM phosphate salt and at about pH 7.1.

The vaccine formulation used for in vivo administration are commonly sterile, which can be accomplished by filtration through sterile filtration membranes, prior to, or following, lyophilization and reconstitution. Alternatively, sterility of the entire composition may be accomplished by autoclaving the ingredients, except for protein, at about 120° C. for up to about 30 minutes, as one non-limiting example.

For the actual lyophilization procedure itself, different commercially devices are available, including Hull50™ (Hull, USA) or GT20™ (Leybold-Heraeus, Germany) BOC Edwards, Christ GTS, FTS Lyostar freeze-dryers. Freeze-drying is accomplished by freezing the formulation and subsequently subliming ice from the frozen content at a temperature suitable for primary drying. Under this condition, the product temperature is below the eutectic point or the collapse temperature of the formulation. Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will mainly dictate the time required for drying, which can range from a few hours to several days (e.g. 40-60 hrs). A secondary drying stage may be carried out at about 0-40° C., depending primarily on the type and size of container and the type of protein employed. However, it was found herein that a secondary drying step may not be necessary. For example, the shelf temperature throughout the entire water removal phase of lyophilization may be from about 15-30° C. (e.g., about 20° C.). The time and pressure required for secondary drying will be that which produces a suitable lyophilized cake, dependent, e.g., on the temperature and other parameters. The secondary drying time is dictated by the desired residual moisture level in the product and typically takes at least about 5 hours (e.g. 10-15 hours). The pressure may be the same as that employed during the primary drying step. Freeze-drying conditions can be varied depending on the formulation and vial size. The resulting lyophilized powder will commonly possess a moisture content thereof that is less than about 5%, and preferably less than about 3%.

Examples of freeze-drying cycles useful in the methods of the invention are shown in Tables 1 and 2:

TABLE 1
Freeze Drying Cycle

| STEP | TIME (h/m | Temp (° C.) | Vac (pB) | Run Time |
|---|---|---|---|---|
| Load | 10 m | | | 10 m |
| Ramp | 4 h | −40 | | 4 h 10 m |
| Hold | 4 h | −40 | | 8 h 10 m |
| Hold | 45 m | −40 | 200 | 8 h 55 m |
| Ramp | 40 m | −30 | 200 | 9 h 35 m |
| Hold | 18 h | −30 | 200 | 27 h 35 m |
| Ramp | 8 h 20 m | 20 | 200 | 35 h 55 m |
| Hold | 4 h | 20 | 200 | 39 h 55 m |
| Ramp | 1 h | 5 | 200 | 40 h 55 m |
| Hold | 35 m | 5 | 200 | 41 h 55 m |
| Stoppering | | 5 | 200 | |

Freeze drying of the samples of Table 1 was accomplished using an LS40 freeze drier obtained from Severn Sciences Ltd.

TABLE 2
Freeze-dry cycle.

| | Time | Temp | Vac |
|---|---|---|---|
| STEP | (Min) | (° C.) | (mTorr) |
| Load | 0 | 20 | |
| Ramp | 240 | −40 | |
| Hold | 180 | −40 | |
| Hold | 45 | −40 | 113 |
| Ramp | 40 | −30 | 113 |
| Hold | 2400 | −30 | 113 |
| Ramp | 500 | 20 | 113 |
| Hold | 480 | 20 | 113 |
| Ramp | 60 | 5 | 113 |
| Hold | 35 | 5 | 113 |
| Stoppering | | 5 | 113 |

This modified cycle of Table 2 was performed using a Christ freeze-dryer.

In each cycle, the product was stoppered under vacuum before introducing air into the chamber. Aluminium seals were then applied to the vials and crimped, on removal from the freeze dryer.

The reconstituted formulation is administered to a patient in need of treatment with the protein, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes.

The lyophilized vaccine compositions prepared by the methods of the invention are typically maintained in the freeze-dried state until the time of use. When ready for administration to a patient, the lyophilized formulation is reconstituted with a diluent as described herein.

Reconstitution commonly occurs at room temperature to ensure complete hydration, although other temperatures may be employed as desired. The time required for reconstitution will depend, e.g., on the type of diluent, amount of excipient(s) and protein, such as rPA. Examples of diluents include sterile water, bacteriostatic water for injection (BWFI), a pH buffered solution (e.g. phosphate-buffered saline is especially preferred), sterile saline solution, Ringer's solution or dextrose solution. The diluent may optionally contain a preservative. Aromatic alcohols such as benzyl or phenol alcohol are preferred preservatives. The amount of preservative employed is determined by assessing different preservative concentrations for compatibility with the protein and preservative efficacy testing. For example, if the preservative is an aromatic alcohol (such as benzyl alcohol), it can be present in an amount from about 0.1-2.0% and preferably from about 0.5-1.5%, but most preferably about 1.0-1.2%.

The present invention further provides a method of protecting against, or a method of treating, a microbial infection, preferably a bacterial infection, comprising administering to a mammal at risk of such infection a therapeutically-effective amount of a stable vaccine formulation as described herein. The infecting organism may be any one or more of those recited herein and the formulation being one recited herein and/or a formulation reconstituted from a vaccine composition or mixture freeze-dried according to the methods of the invention. In a preferred embodiment, the bacterial infection is an anthrax infection (i.e., caused by Bacillus anthracis). In one example, the animal to be protected is a mammal, especially a human patient. A preferred formulation for use in such treatment is a vaccine composition containing 200 μg/ml rPA, 2 mg/ml CpG1018, 5% trehalose, 5.25 mM phosphate, 0.39% NaCl, 0.02% Tween 20, 0.26% w/v alhydrogel.

In carrying out the procedures of the present invention it is of course to be understood that reference to particular buffers, media, reagents, and the like are not intended to be limiting, but are to be read so as to include all related materials that one of ordinary skill in the art would recognize as being of interest or value in the particular context in which that discussion is presented. For example, it is often possible to substitute one buffer system for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein, where said materials are not essential to the invention.

The invention is described in more detail in the following non-limiting example. It is to be understood that these methods and examples in no way limit the invention to the embodiments described herein and that other embodiments and uses will no doubt suggest themselves to those skilled in the art.

### EXAMPLE 1

### Stability of Trehalose-Containing Lyophilized Vaccine

400 μg/ml rPA, 4 mg/ml CpG1018 in 10% trehalose, 0.5 mM phosphate, 10 mM NaCl, 0.04% Tween 20 was lyophilized. This formulation composition prior to lyophilization is twice the concentration of the reconstituted material. This formulation was then reconstituted with 0.26% alhydrogel in 5 mM phosphate, 0.39% saline, pH 7.4. The reconstituted formulation was 200 μg/ml rPA, 2 mg/ml CpG1018 in 5% trehalose, 5.25 mM phosphate, 0.39% NaCl, 0.02% Tween 20, 0.26% alhydrogel. All % values are w/v.

At 70° C., rPA retained potency after up to 8 months. Because 70° C. is above the Tg (glass transition temperature, where the trehalose sugar glass starts to soften and is no longer brittle) it would have been expected to find increased degradation but no such increased degradation was observed. Thus, this lyophilized formulation was unexpectedly stable in the presence of trehalose.

### EXAMPLE 2

### Stability of the Lyophilized Vaccine

Female adult A/J mice were immunized on day 0 intramuscularly (i.m.) with 0.1 ml of the respective samples indicated in Table 3, which were formulated with diluent prior to administration. All the mice were challenged with B. anthracis STI strain spores on day 21 and survival was determined on day 11.

The results of Table 3 show a much greater stability for a lyophilized vaccine than would have been expected based on previous experience with such vaccines (compare, for example, the results for storage at 70° C. with that for 5° C.). The diluent was comprised of: 0.26% (w/w) alhydrogel (0.4% Al(OH)₃) in 0.39% (w/v) sodium chloride buffered with 5 mM phosphate, pH 7.4. The naïve animals were negative controls that had not received any treatment but were challenged at the day 21 with the same dose of spores as the test animals.

TABLE 3
Results of immunization following storage.

| | Storage | | |
|---|---|---|---|
| | Temperature | Storage Time | Challenge Survival |
| Sample | (° C.) | (months) | (day 11) |
| Lyo rPA/trehalose + | 5° C. | 16 | 10/10 |
| diluent | | | |
| (lot#050207A) | | | |
| Lyo rPA/trehalose + | 55° C. | 16 | 10/10 |
| diluent | | | |
| (lot#050207A) | | | |
| Lyo rPA/trehalose + | 5° C. | 7 | 10/10 |
| diluent | | | |
| (lot#290807A) | | | |
| Lyo rPA/trehalose + | 70° C. | 7 | 10/10 |
| diluent | | | |
| (lot#290807A) | | | |
| Naïve | N/A | N/A | 0/5 |

What is claimed:

1. A stable reconstituted vaccine formulation, comprising an anthrax antigen and a pharmaceutically acceptable carrier, which reconstituted formulation has been prepared from a lyophilized composition of said anthrax antigen and a lyoprotective amount of a non-reducing sugar.

2. The stable reconstituted vaccine formulation of claim 1, wherein said anthrax antigen contains recombinant anthrax Protective Antigen (rPA).

3. The stable reconstituted vaccine formulation of claim 2, wherein said formulation retains substantially the same potency as said composition prior to said lyophilization.

4. The stable reconstituted vaccine formulation of claim 1, wherein said formulation retains at least 80% of the potency of said composition prior to said lyophilization.

5. The stable reconstituted vaccine formulation of claim 1, wherein said formulation retains at least 85% of the potency of said composition prior to said lyophilization.

6. The stable reconstituted vaccine formulation of claim 1, wherein said formulation retains at least 90% of the potency of said composition prior to said lyophilization.

7. The stable reconstituted vaccine formulation of claim 1, wherein said formulation retains at least 95% of the potency of said composition prior to said lyophilization.

8. The stable reconstituted vaccine formulation of claim 3, wherein said rPA is present in said reconstituted formulation in the range of about 100 to 300 μg/ml.

9. The stable reconstituted vaccine formulation of claim 3, wherein said rPA is present in said reconstituted formulation in the range of about 150 to 250 μg/ml.

10. The stable reconstituted vaccine formulation of claim 3, wherein said rPA is present in said reconstituted formulation at about 200 μg/ml.

11. The stable reconstituted vaccine formulation of claim 3, wherein said non-reducing sugar is trehalose.

12. The stable reconstituted vaccine formulation of claim 11, wherein said trehalose is present in said reconstituted formulation in the range of about 3% to 7% (w/v).

13. The stable reconstituted vaccine formulation of claim 11, wherein said trehalose is present in the range of about 4% to 6% (w/v).

14. The stable reconstituted vaccine formulation of claim 11, wherein said trehalose is present in the range of about 4.5% to 5.5% (w/v).

15. The stable reconstituted vaccine formulation of claim 11, wherein said trehalose is present in said reconstituted formulation at about 5% (w/v).

16. The stable reconstituted vaccine formulation of claim 3, wherein said lyophilized composition was maintained at a temperature of at least 25° C. for a period of at least 3 months prior to reconstitution.

17. The stable reconstituted vaccine formulation of claim 16, wherein said temperature is at least 40° C.

18. The stable reconstituted vaccine formulation of claim 16, wherein said temperature is at least 55° C.

19. The stable reconstituted vaccine formulation of claim 16, wherein said temperature is at least 70° C.

20. The stable reconstituted vaccine formulation of claim 16, wherein said period is at least 3 months.

21. The stable reconstituted vaccine formulation of claim 16, wherein said period is at least 7 months.

22. The stable reconstituted vaccine formulation of claim 16, wherein said period is at least 8 months.

23. The stable reconstituted vaccine formulation of claim 16, wherein said period is at least 16 months.

24. The stable reconstituted vaccine formulation of claim 3, wherein said stable formulation comprises an adjuvant.

25. The stable reconstituted vaccine formulation of claim 24, wherein said adjuvant is selected from alhydrogel (alum), chitosan, mpl and CpG.

26. The stable reconstituted vaccine formulation of claim 24, wherein said adjuvant is alhydrogel (alum).

27. The stable reconstituted vaccine formulation of claim 3, wherein said reconstituted formulation comprises 200 μg/ml rPA, 2 mg/ml CpG1018, 5% trehalose, 5.25 mM phosphate, 0.39% NaCl, 0.02% Tween 20, 0.26% w/v alhydrogel.

28. A method for preparing a stable lyophilized vaccine formulation, comprising the steps of:
(a) forming a composition of an anti-microbial antigen and a lyoprotective amount of a reducing sugar; and
(b) freeze-drying said composition to form a stable lyophilized vaccine formulation.

29. The method of claim 28, wherein said anti-microbial antigen is rPA.

30. The method of claim 28, wherein said non-reducing sugar is trehalose.

31. A method of protecting against a microbial infection, comprising administering to a mammal at risk of such infection a therapeutically-effective amount of the stable reconstituted vaccine formulation of claim 1.

32. The method of claim 31, wherein said microbial infection is an anthrax infection and said anti-microbial antigen is an anthrax antigen.

33. The method of claim 32, wherein said anti-microbial antigen is rPA.

34. The method of claim 33, wherein said mammal is a human being.

35. The method of claim 33, wherein said stable reconstituted formulation comprises 200 μg/ml rPA, 2 mg/ml CpG1018, 5% trehalose, 5.25 mM phosphate, 0.39% NaCl, 0.02% Tween 20, 0.26% w/v alhydrogel.
